(19) United States
(12) Patent Application Publication
(10) Pub. No.: US 2022/0277839 A1
(43) Pub. Date: Sep. 1, 2022

(54) MODEL TO DYNAMICALLY PREDICT PATIENT'S DISCHARGE READINESS IN GENERAL WARD

(71) Applicant: KONINKLIJKE PHILIPS N.V., EINDHOVEN (NL)

(72) Inventors: Shruti Gopal Vij, Cambridge, MA (US); Gregory Boverman, Cambridge, MA (US); David Paul Noren, Cambridge, MA (US); Lasith Adhikari, Cambridge, MA (US); Jochen Weichert, Cambridge, MA (US); Jeanne Cheng, Cambridge, MA (US)

(21) Appl. No.: 17/627,137

(22) PCT Filed: Jul. 13, 2020
(86) PCT No.: PCT/EP2020/069681

§ 371 Date: Jan. 14, 2022

Related U.S. Application Data
(60) Provisional application No. 62/874,112, filed on Jul. 15, 2019.

(51) Int. Cl. G16H 40/20; G16H 10/60; G16H 50/30; G06N 20/00

(52) U.S. Cl. CPC G16H 40/20; G16H 10/60; G16H 50/30; G06N 20/00

(57) ABSTRACT

A method for identifying patients for discharge from a general ward in a hospital, including: calculating a transition score of a patient based upon patient vital sign information; computing a TS upper bound value and a TS lower bound value based upon a set of TS values in a TS time window; determining if a length of stay of the patient is greater than a first time window, greater than an expected length of stay, and greater than a lower evaluation window; determining if a current TS lower bound value is less than a lower threshold; and producing an indication that that the patient is to be evaluated for discharge from the general ward when it is determined that the length of stay of the patient is greater than the first time window, greater than the expected length of stay, and greater than the lower evaluation window and that the current TS lower bound value is less than the lower threshold.

### TECHNICAL FIELD

Various exemplary embodiments disclosed herein relate generally to a model to dynamically predict patient's discharge readiness in general ward.

### BACKGROUND

Innovative technologies in diagnostic and therapeutic procedures have been consistently on the rise in the last decade. The increased knowledge of available technologies through the internet and social media has resulted in an increased demand for hospitalization and medical support as well as higher quality of healthcare services. Assessing patient discharge readiness is a significant factor for the hospitals to keep up with the demand for healthcare services. Accurate estimates of when a patient will be discharged help hospitals to better manage resources and to better understand their peak capacity. This also allows for various steps related to discharge to be started ahead of time, for example, the next point of care or contacting a family member to help get the patient home. While there has been significant research in assessing the discharge readiness for a patient from the ICU to the general ward, little research has been done to predict the length of stay of a patient in the general ward. See Badawi, Omar, and Michael J. Breslow. “Readmissions and death after ICU discharge: development and validation of two predictive models.” PloS one 7.11 (2012): e48758 and Badawi, Omar. “Discharge readiness index.” U.S. patent application Ser. No. 14/125,327.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method for identifying patients for discharge from a general ward in a hospital, including: calculating a transition score of a patient based upon patient vital sign information; computing a TS upper bound value and a TS lower bound value based upon a set of TS values in a TS time window; determining if a length of stay of the patient is greater than a first time window, greater than an expected length of stay, and greater than a lower evaluation window; determining if a current TS lower bound value is less than a lower threshold; and producing an indication that that the patient is to be evaluated for discharge from the general ward when it is determined that the length of stay of the patient is greater than the first time window, greater than the expected length of stay, and greater than the lower evaluation window and that the current TS lower bound value is less than the lower threshold.

Various embodiments are described, further including producing no recommendation regarding patient discharge when it is determined that the length of stay of the patient is not greater than a first time window, not greater than and expected length of stay, or not greater than a lower evaluation window.

Various embodiments are described, further including producing no recommendation regarding patient discharge when it is determined that that the current TS lower bound value is not less than the lower threshold.

Various embodiments are described, wherein the first time window has a value in the range of 8 to 24 hours.

Various embodiments are described, wherein the values of the first window, the lower evaluation window, and the lower threshold are determined by using machine learning techniques with patient training data.

Various embodiments are described, wherein the transition scores is further based upon diagnostic results, procedures performed, drugs consumed, medical images, or patient demographic information.

Various embodiments are described, wherein the patient vital signs include heart rate, respiration rate, peripheral capillary oxygen saturation (SpO2), blood pressure, and temperature.

Various embodiments are described, wherein calculating a transition score of a patient based upon patient vital sign information only occurs when the vital signs were measure within a specified recent period of time.

Various embodiments are described, further including: determining if a length of stay of the patient is greater than a second time window and greater than an upper evaluation window; determining if a current TS upper bound value is greater than an upper threshold; and producing an indication that that the patient is to be evaluated for a step-up transition from the general ward when it is determined that the length of stay of the patient is greater than the second time window and greater than the lower evaluation window and that the current TS lower bound value is greater than the upper threshold.

Various embodiments are described, wherein the values of the second window, the upper evaluation window, and the upper threshold are determined by using machine learning techniques with patient training data.

Further various embodiments relate to a non-transitory machine-readable storage medium encoded with instructions for identifying patients for discharge from a general ward in a hospital, comprising instructions for: calculating a transition score of a patient based upon patient vital sign information; computing a TS upper bound value and a TS lower bound value based upon a set of TS values in a TS time window; determining if a length of stay of the patient is greater than a first time window, greater than and expected length of stay, and greater than a lower evaluation window;

determining if a current TS lower bound value is less than a lower threshold; and producing an indication that that the patient is to be evaluated for discharge from the general ward when it is determined that the length of stay of the patient is greater than the first time window, greater than the expected length of stay, and greater than the lower evaluation window and that the current TS lower bound value is less than the lower threshold.

Various embodiments are described, further including instructions for producing no recommendation regarding patient discharge when it is determined that the length of stay of the patient is not greater than a first time window, not greater than and expected length of stay, or not greater than a lower evaluation window.

Various embodiments are described, further including instructions for producing no recommendation regarding patient discharge when it is determined that that the current TS lower bound value is not less than the lower threshold.

Various embodiments are described, wherein the first time window has a value in the range of 8 to 24 hours.

Various embodiments are described, wherein the values of the first window, the lower evaluation window, and the lower threshold are determined by using machine learning techniques with patient training data.

Various embodiments are described, wherein the transition scores is further based upon diagnostic results, procedures performed, drugs consumed, medical images, or patient demographic information.

Various embodiments are described, wherein the patient vital signs include heart rate, respiration rate, peripheral capillary oxygen saturation (SpO2), blood pressure, and temperature.

Various embodiments are described, wherein calculating a transition score of a patient based upon patient vital sign information only occurs when the vital signs were measure within a specified recent period of time.

Various embodiments are described, further including instructions for: determining if a length of stay of the patient is greater than a second time window and greater than an upper evaluation window; determining if a current TS upper bound value is greater than an upper threshold; and producing an indication that that the patient is to be evaluated for a step-up transition from the general ward when it is determined that the length of stay of the patient is greater than the second time window and greater than the lower evaluation window and that the current TS lower bound value is greater than the upper threshold.

Various embodiments are described, wherein the values of the second window, the upper evaluation window, and the upper threshold are determined by using machine learning techniques with patient training data.

### BRIEF DESCRIPTION OF THE DRAWINGS

In order to better understand various exemplary embodiments, reference is made to the accompanying drawings, wherein:

FIG. 1 illustrates the typical length-of-stay probability distribution for patients in a general ward;

FIG. 2 illustrates a tornado plot showing relative importance of features in predicting discharge in 24 hours;

FIG. 3 illustrates the performance of the multivariate logistic regression classifier;

FIG. 4 illustrates the ROC curve for predicting patient deterioration using the early deterioration index (EDI);

FIG. 5 illustrates positive-predictive value or precision versus upper bound of percentage of bed days saved for multiple median filter windows;

FIG. 6 illustrates an algorithm for computing the discharge/deterioration transition score given time-series vital signs with some values potentially missing and for computing the upper and lower bounds for transition evaluation and visualization;

FIG. 7 illustrates a discharge recommendation algorithm for recommending discharge, evaluations for a step-up transition, or no recommendation based on the transition score (TS) and the lower and upper TS bounds; and

FIG. 8 illustrates a block diagram for a patient discharge recommendation system.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

Hospital resource utilization is dependent on patients' length of stay in the general ward. Patient discharge readiness is a quantitative mechanism to predict how long the patient will need to receive medical services before being declared stable enough to be discharged home. However, there has been no known discharge prediction algorithm for the general ward. An embodiment of a clinical decision support tool used by nurse managers and hospitalists in the general ward to assist in discharging a patient will be described herein. Accurately predicting a patient's discharge readiness in the general ward is critical for many factors such as, inter alia, 1) reducing costs for the hospital, 2) reduce early discharges that might be resulting in readmissions within 30 days, and 3) reducing the incidence of hospital acquired infections in cases where patients are not being promptly discharged. Additionally, the embodiment described herein combine a stability assessment of a patient with a discharge prediction model. The prediction model will make use of a number of possible machine-learning algorithms including as examples decision trees, random forests, support vector machines, neural networks, and recurrent neural networks. These algorithms aim to effectively model non-linear relationships between patient factors and the percentage probability of discharge of the patient.

The predictive model has two main elements to it: 1) using previously collected vital signs to assess patient's stability; and 2) using this stability index and early warning scores (EWS) to predict whether the patient should be discharged in the next 12 hours.

First, a multi-level assessment of patient's stability is determined using all the vital signs—BP, HR, RR, Temperature and SpO2 saturation. Additional or fewer vital signs may be used as well in this assessment. Adding a time factor to this assessment may allow for the assessment of how long the patient has had stable vital signs. The stability assessment may be conducted by assessing a patient's historical vital scores from the beginning of admission to the ward in 24 hour increments to quantify adverse event incidences relating to any of the important vital signs in a comparable time-package. Other time increments may also be used in this stability assessment. The greater the length of time the patient has been stable, the better the stability score will be. An assessment of instability will result in a lower scoring for the stability measure. The uniqueness of this patient stability measure is that it will use aggregated information for the patient in 24 hr increments instead of instantaneous information as the EWS score does. Secondly, a non-linear data driven predictive model is used that combines the above estimated stability score with other demographic information and health record data such as ICD-9 diagnostic code, current vital signs measurements or current EWS score, and how long the patient has currently been in the ward to predict if the patient should be discharged from the general ward in the near future, for example within the next 12 hours.

The simplest approach is based on analysis of typical patterns of lengths of stay. Secondly, an algorithm for recognizing a patient's readiness for discharge based on past hospital practices is described. This approach achieved modest predictive accuracy in the task of determining whether a patient will be discharged in a time window in the near future, for example 24 hours in the near future. Lastly, the predictive accuracy of an algorithm that detects whether a patient will experience a deterioration at any point in the future is described and evaluated. In the description herein of discharge from the general ward, the Early Deterioration Index (EDI) as a measure of a patient's acuity is used. The EDI is described in detail in E. Ghosh, L. Eshelman, L. Yang, E. Carlson, and B. Lord, “Early Deterioration Indicator: Data-driven approach to detecting deterioration in general ward,” Resuscitation, vol. 122, pp. 99-105, 2018, which is incorporated herein by reference for all purposes as if included herein. The EDI uses a small set of vital signs and develops a model regarding the deterioration of a patient. Further, while the EDI is used as a detection metric other metrics may also be used in the embodiments described herein.

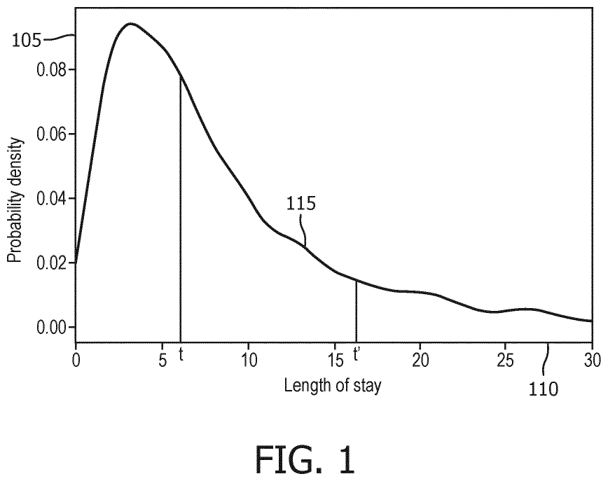

In the simplest approach, a model, either parametric or non-parametric, of the probability distribution of a patient's length-of-stay in a ward is developed. Given that a patient has already stayed for a length of time t, the conditional distribution of any additional time period, t′, can be computed either by symbolic or numerical integration. FIG. 1 illustrates the typical length-of-stay probability distribution for patients in a general ward. The vertical axis 105 of the plot is a measure of the probability density of patients for each length of stay. The horizontal axis 110 is the length of stay in days. The plot 115 shows the probability density of the patient's length of stay for each time. The conditional distribution for the additional time period t′ from time t is shown in FIG. 1 which corresponds to the area under the curve 115 between time t and t′.

This approach produces results that are linked to the very specific probability distribution curve, which does not produce as accurate of results as may be desired. Therefore, a machine-learning model will now be described for the prediction of discharge.

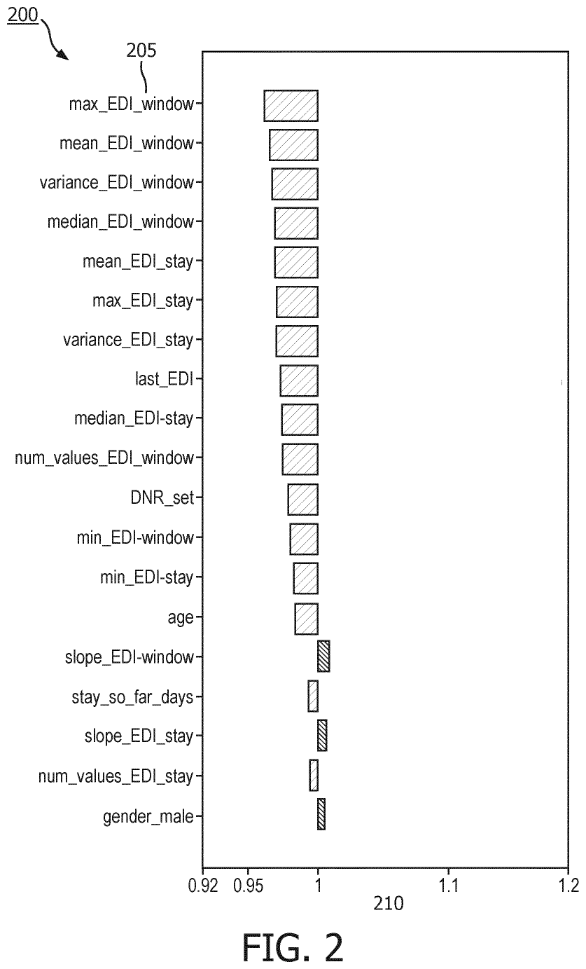

Methods for prediction of discharge in the near future based on machine-learning techniques will now be described. Specifically, at each point in time, a feature vector is created that is composed of derived metrics from the time series of EDI values in the recent past, as defined by some time “window”. Various time windows may be used. This feature vector contains such features as the maximum, mean, variance of the EDI values within the window as well of the entire stay to date. FIG. 2 illustrates a tornado plot showing relative importance of input features in predicting discharge in 24 hours. The feature vector 205 includes various features such as maximum, minimum, mean, variance, slope, and mean EDI values during the time window. For example, the time window may be 24 hours, but other values may be used. Also mean, maximum, minimum, median, slope, and variance EDI values for the complete stay may be features. Further, the last EDI value, the number of EDI values in the window, the presence of a do not resuscitate order, age, length of stay, the number of EDI values in the stay, and gender may also be features. Other features not described may also be used. The outcome may be defined as hospital discharge in the next 24 hours. The features near the top show greater indication that these features influence the likelihood of discharge, while those near the bottom show little effect on the likelihood of discharge.

The tornado plot of FIG. 2 displays the relative importance of these derived time-series parameters in predicting discharge, as computed by univariate logistic regression with the odds ratio displayed for each feature along the horizontal axis 210. As one would expect, the maximum value of the EDI in a short time window preceding the current time is negatively correlated with near-term discharge, although the odds ratio of the relationship is not large. The tornado plot illustrates that the use of a time window was helpful, with a stronger effect (odds ratio differing more greatly than one) for the derived parameters than for the current value of the EDI score itself (denoted by “last_EDI”).

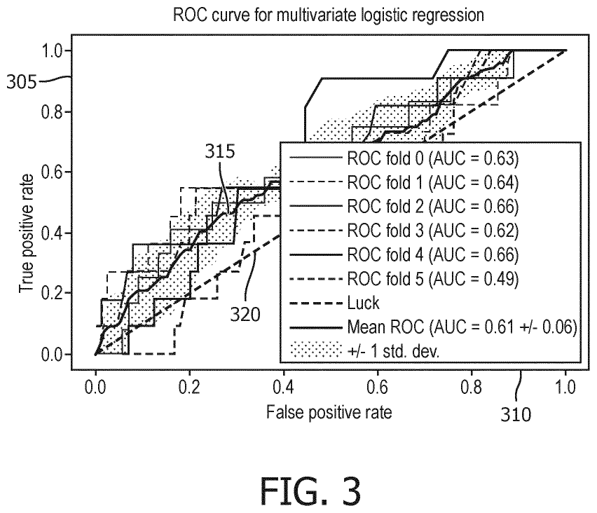

Given this set of feature vectors, the time-series prediction problem may be solved using a number of techniques, for example, multivariate logistic regression and random forests, but other machine learning techniques may be used as well. The performance of the multivariate logistic regression classifier is shown in FIG. 3, which indicates that only modest prediction performance is possible using vital signs alone. In FIG. 3, the receive operating characteristic (ROC) curves are shown for a various area under the curve (AUC) values. The vertical axis 305 indicate the true positive rate, and the horizontal axis indicates the false positive rate. The mean ROC curve 315 is shown. The curve 320 shows the plot for luck where correct value is guessed. The difference between the mean ROC curve 315 and the luck curve 320 shows the modest prediction performance. It is noted that performance may be significantly improved by including features related to patient diagnosis and to clinical events, such as tests performed, and lab results. So, this leads to the fact that while stable vital signs are important in determining whether a patient should be discharged, additional information will help refine a determination of when the patient is ready to be discharged.

In the previous section, an outcome that is typically subjective in nature, the decision to discharge a patient, was considered. Now a more objective outcome will be considered, that is, prediction of patients not likely to deteriorate in the future.

In order to accomplish this goal, first a methodology to predict patient deterioration is developed, which in the general ward is defined as a patient either dying shortly after his or her hospital stay or transfer to a higher level of acuity, for example the intensive care unit (ICU). To some extent, the prediction of deterioration as measured by EDI was discussed above, but here the time-series prediction problem is considered. The use of the EDI over time helps to improve the ability to predict when a patient may be discharged because they have stabilized.

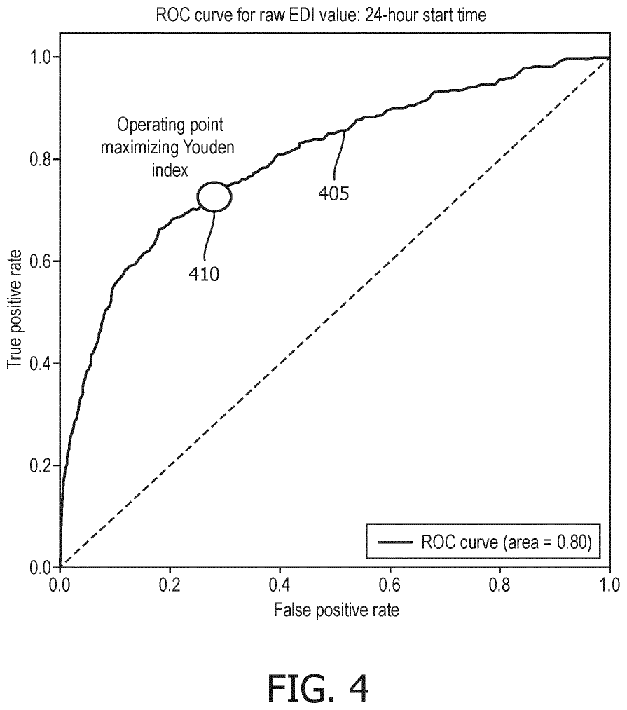

It is proposed to predict deterioration using a particular threshold of the raw EDI score, as shown in FIG. 4. In FIG. 4 an operating point 410 is selected as the threshold maximizing the Youden index, which optimally balances sensitivity and specificity. Specifically, FIG. 4 illustrates the ROC curve for predicting patient deterioration using the early deterioration index (EDI). The ROC curve 405 plots the true positive rate versus false positive rate for the EDI. The detection performance of this operating point is shown in Table 1. So various EDI thresholds are evaluated and then the one with the lowest Youden index is chosen. Other performance metrics may also be used to determine the optimum operating point.

TABLE 1

| Parameter for Maximizing | |
|---|---|
| Youden Index | Value |
| EDI Threshold | 28 of a maximum of 100 |
| Sensitivity | 66% |
| Specificity | 82% |
| Number of True Positives | 227 |
| Positive predictive value | 15% |

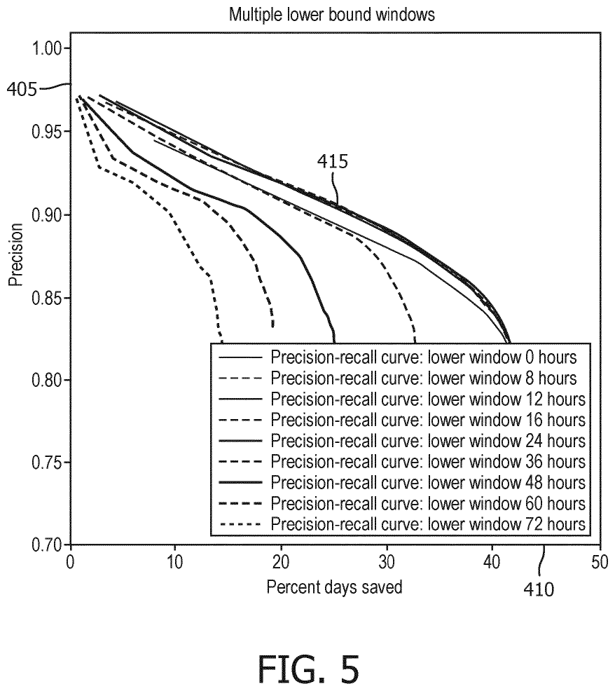

Once the threshold for deterioration has been found, machine learning and signal processing techniques may be used to determine optimal parameters for predicting patient near-term stability. In the case of predicting patient deterioration, it was determined that temporal filtering of the EDI time-series was not helpful in improving prediction performance, but in the case of patient deterioration, it was helpful to consider patient stability over time. The experimental performance is shown in FIG. 5 which illustrates positive-predictive value or precision versus upper bound of percentage of bed days saved for multiple median filter windows. The plot illustrates that the curves for windows of sizes 8-24 hours are above that for a window of 0 hours (just looking at the current value) and therefore it is beneficial to use some median filter smoothing. The percentage of days saved was calculated by determining that a true positive was detected when a patient reached the stability condition and never subsequently deteriorated or reached the EDI threshold indicative of deterioration, as shown in Table 1. If a patient did deteriorate, it was determined that there were no bed days saved.

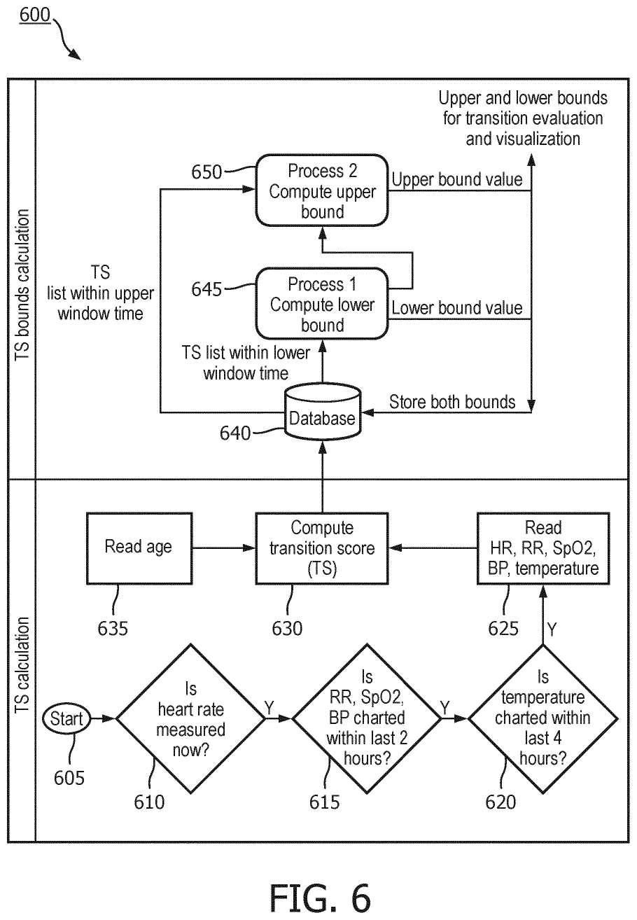

FIG. 6 illustrates an algorithm for computing the discharge/deterioration transition score (TS) given time-series vital signs with some values potentially missing and for computing the upper and lower bounds for transition evaluation and visualization. The TS score is based upon evaluating the value of the EDI over time. The TS score may also be based upon other deterioration metrics as well. This algorithm may be a patient stability estimator. The algorithm 600 begins 605 and the determines if the patient's heart rate (HR) has been measured 610. If so, then the algorithm 600 determines if respiration rate (RR), peripheral capillary oxygen saturation (SpO2), and blood pressure (BP) have been charted in the last two hours 615. Note that other time windows may be used here as well. If so, the algorithm 600 then determines if the patient's temperature 620 has been charted within the last four hours. Again, note that other time windows may be used as well. Next, the algorithm 600 reads the HR, RR, SpO2, and BP 625 as well as obtaining the patient's age 635. These first steps show that the TS is only going to be computed if recent measurements are available because TS values calculated using older data become less reliable and accurate. The algorithm 600 uses all of this information the compute the TS 630 which may be stored in the database 640. Next, the algorithm uses a first process 645 to calculate a lower TS bound using a list of TS values within a lower window time. The algorithm also uses a second process 650 to calculate an upper bound using a list of TS values within the upper window time. A related patent application 62/802,733 filed on Feb. 8, 2019, which is incorporated herein for all purposes as if fully disclosed herein, describes one method for calculating these upper and lower TS bounds. Other methods may also be used to determine the lower and upper TS bounds. Upper and lower TS bounds computed by the first process 645 and the second process 650 are then used in a discharge recommendation algorithm to determine whether it is recommended to discharge the patient, to evaluate for a step-up transition, or that there is no recommendation regarding the discharge of the patient at this time. The effect of this is to determine if the patient has been stable over a specific period of time, which indicates that they are likely not going to deteriorate if they are discharged from the general ward. It will also help indicate ultimately if the patient needs to be stepped-up to more acute care.

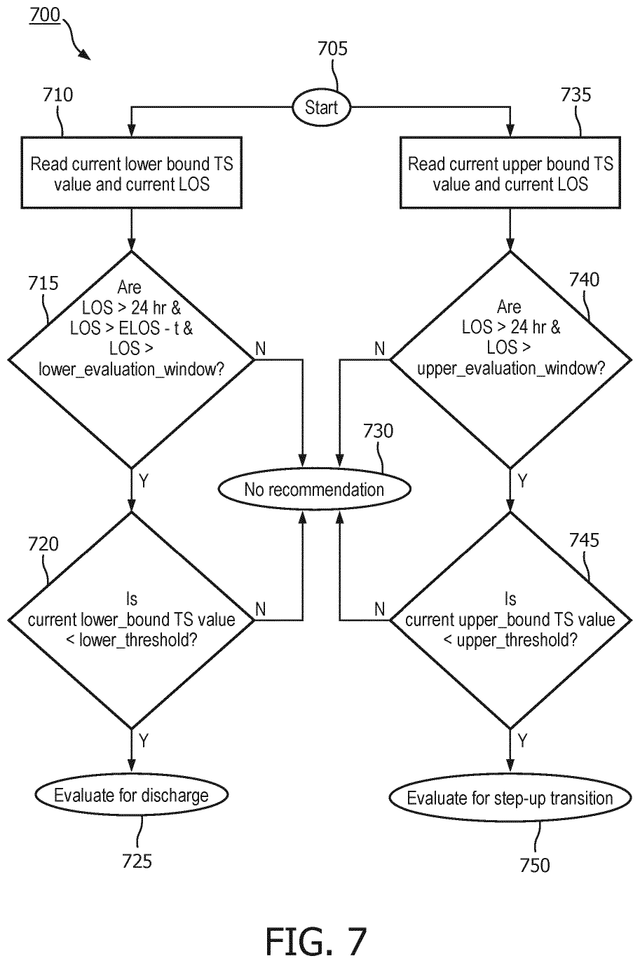

FIG. 7 illustrates a discharge recommendation algorithm for recommending evaluation for discharge, evaluations for a step-up transition, or no recommendation based on the TS and the lower and upper TS bounds. The discharge recommendation algorithm 700 begins at 705. The algorithm has two paths based upon the lower TS bound value and the upper TS bound value. The algorithm reads the current lower TS bound value and the current length of stay (LOS) 710. The LOS value is then used to do the following comparison: LOS>24 hours & LOS>ELOS−t & LOS>lower_evaluation_window 715. Here the limit of 24 hours may be considered a first time window that may have other values as well. The first time window could also be in the ranges of 8 to 24 hours, 24 to 35 hours, or 8 to 36 hours. ELOS is expected length of stay for various types of procedures, and t is a window of time denoting when to start evaluating the patient stability. For example, for a knee surgery the value oft may be 2 days, but for a hip replacement it may be 4 days. The difference between them illustrates the point in time where it is reasonable to start to consider whether to start evaluating the patient as a candidate for discharge. The lower_evaluation_window puts a lower bound on the LOS. If the LOS does not meet all of these conditions, then no recommendation is made 730. If the LOS does meet all of these conditions, then the algorithm 700 determines if the current lower TS bound value is less than a lower threshold value 720. If not, then the no recommendation is made 730. If so, then the algorithm recommends that the patient be evaluated for discharge 725.

From this algorithm, it is noted that the size of the window needs to be balanced. Too short of a window and patients may be sent home early that may later deteriorate with negative consequences. On the other hand, if the window is too large, the patient stays in the hospital longer than needed which adds to cost may increases the patient's exposure to hospital borne infections.

The second path of the algorithm 700 will now be described. The algorithm reads the current upper TS bound value and the current LOS 735. The LOS value is then used to do the following comparison: LOS>24 hours & LOS>upper_evaluation_window 740. Here the limit of 24 hours may be considered a second time window that may have other values as well. The first and second time windows may have the same or different values. The upper_evaluation_window puts an upper bound on the LOS. If the LOS does not meet all of these conditions, then no recommendation is made 730. If the LOS does meet all of these conditions, then the algorithm 700 determines if the current upper TS bound values is greater than an upper threshold value 745. If not, then the no recommendation is made 730. If so, then the algorithm recommends that the patient be evaluated for a step-up transition 750.

The various values used in the algorithm 700 may be developed using machine learning techniques. For example, the values for the different windows sizes, lower_evaluation_window, upper_evaluation_window, lower_threshold, and upper_threshold may be determined in this way. This may be done by picking a set of values for each of the parameters and then doing an exhaustive search to determine which values providing the best performance. Such variation in these parameters are shown in FIG. 5. A performance metric may be the area under one of the curves in FIG. 5, but other performance metrics may be used as well. Further, gradient decent and other machine learning techniques may be used to vary the different parameters until an optimal set of parameters is found. This training and optimization of the algorithms may be done using collected patient data where the outcome is known.

It is noted that these models may be developed to be trained based upon various types of patients and facilities. For example, the model may be specific to a certain hospital. In other embodiments, the model may be trained for cardiac patients, orthopedic patients, as well as more specific procedures, etc.

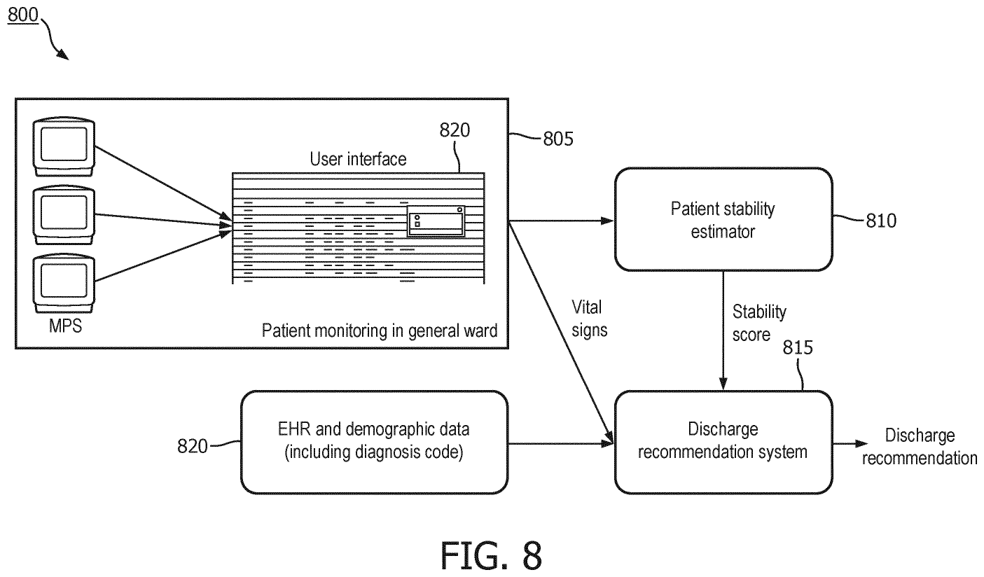

FIG. 8 illustrates a block diagram for a patient discharge recommendation system. The patient discharge recommendation system 800 may include patient monitoring system 805 having a user interface 820. The user interface 820 may be implemented on desktop or laptop computers, mobile phones, tablets, or any other device capable of providing a user interface to a user. The user interface 820 allows a user, such as a medical professional, to evaluate and monitor patient's readiness to be discharged from the general ward or to be moved to a more acute care ward such as an intensive care unit. The patient monitoring system 805 may also collect patient vital sign information and make that vital sign information available to other parts of the system. The patient discharge recommendation system 810 may also include a database 820 that includes electronic health records (EHR) and demographic information for patients. Such information may include diagnostic test data, imaging data, lab results, information regarding procedures, etc. The patient discharge recommendation system 800 includes a patient stability estimator 810 which determines the stability of each patient and outputs a patient stability score. The algorithm 700 described above may be used by the patient stability estimator 810. A discharge recommendation system 815 uses the stability score from the patient stability estimator 810, vital sign data, EHR data, and demographic date for the patient to provide a recommendation regarding whether to discharge a patient from the general ward. FIG. 7 describes a patient discharge algorithm that may be used by the discharge recommendation system 815. Such a recommendation may be updated at periodic intervals, upon the receipt of new information that may affect the recommendation, or upon the request of a user. The output of the discharge recommendation system may include no recommendation, evaluate for discharge, or evaluate for movement to a more acute care ward. This recommendation may be presented to the user on the user interface 820.

The embodiments of a patient discharge recommendation system described herein solve the technological problem of determining when a patient should be considered for discharge from the general ward. Rather than just using prior data regarding patient discharges from the general ward, a model is used to determine a patient transition score. This transition score is based upon the patient's stability. This transition score along with upper and lower bounds are then used to make a recommendation regarding whether the patient should be evaluated for discharge or movement to a more acute ward. This approach improves upon current systems based upon care giver judgement by using measure patient data to determine the patient stability that then leads to a patient transition score that is used to provide the discharge recommendation.

The embodiments described herein may be implemented as software running on a processor with an associated memory and storage. The processor may be any hardware device capable of executing instructions stored in memory or storage or otherwise processing data. As such, the processor may include a microprocessor, field programmable gate array (FPGA), application-specific integrated circuit (ASIC), graphics processing units (GPU), specialized neural network processors, cloud computing systems, or other similar devices.

The memory may include various memories such as, for example L1, L2, or L3 cache or system memory. As such, the memory may include static random-access memory (SRAM), dynamic RAM (DRAM), flash memory, read only memory (ROM), or other similar memory devices.

The storage may include one or more machine-readable storage media such as read-only memory (ROM), random-access memory (RAM), magnetic disk storage media, optical storage media, flash-memory devices, or similar storage media. In various embodiments, the storage may store instructions for execution by the processor or data upon with the processor may operate. This software may implement the various embodiments described above.

Further such embodiments may be implemented on multiprocessor computer systems, distributed computer systems, and cloud computing systems. For example, the embodiments may be implemented as software on a server, a specific computer, on a cloud computing, or other computing platform.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method for identifying patients for discharge from a general ward in a hospital, comprising:
calculating, by a processor, a transition score of a patient based upon patient vital sign information;
computing a TS upper bound value and a TS lower bound value based upon a set of TS values in a TS time window;
determining if a length of stay of the patient is greater than a first time window, greater than an expected length of stay, and greater than a lower evaluation window;
determining if a current TS lower bound value is less than a lower threshold; and
producing an indication that that the patient is to be evaluated for discharge from the general ward when it is determined that the length of stay of the patient is greater than the first time window, greater than the expected length of stay, and greater than the lower evaluation window and that the current TS lower bound value is less than the lower threshold.

2. The method of claim 1, further comprising producing no recommendation regarding patient discharge when it is determined that the length of stay of the patient is not greater than a first time window, not greater than and expected length of stay, or not greater than a lower evaluation window.

3. The method of claim 1, further comprising producing no recommendation regarding patient discharge when it is determined that that the current TS lower bound value is not less than the lower threshold.

4. The method of claim 1, wherein the first time window has a value in the range of 8 to 24 hours.

5. The method of claim 1, wherein the values of the first window, the lower evaluation window, and the lower threshold are determined by using machine learning techniques with patient training data.

6. The method of claim 1, wherein the transition scores is further based upon diagnostic results, procedures performed, drugs consumed, medical images, or patient demographic information.

7. The method of claim 1, wherein the patient vital signs include heart rate, respiration rate, peripheral capillary oxygen saturation (SpO2), blood pressure, and temperature.

8. The method of claim 1, wherein calculating a transition score of a patient based upon patient vital sign information only occurs when the vital signs were measure within a specified recent period of time.

9. The method of claim 1, further comprising:
determining if a length of stay of the patient is greater than a second time window and greater than an upper evaluation window;
determining if a current TS upper bound value is greater than an upper threshold; and
producing an indication that that the patient is to be evaluated for a step-up transition from the general ward when it is determined that the length of stay of the patient is greater than the second time window and greater than the lower evaluation window and that the current TS lower bound value is greater than the upper threshold.

10. The method of claim 9, wherein the values of the second window, the upper evaluation window, and the upper threshold are determined by using machine learning techniques with patient training data.

11. A non-transitory machine-readable storage medium encoded with instructions for identifying patients for discharge from a general ward in a hospital, comprising instructions for:
calculating a transition score of a patient based upon patient vital sign information;
computing a TS upper bound value and a TS lower bound value based upon a set of TS values in a TS time window;
determining if a length of stay of the patient is greater than a first time window, greater than and expected length of stay, and greater than a lower evaluation window;
determining if a current TS lower bound value is less than a lower threshold; and
producing an indication that that the patient is to be evaluated for discharge from the general ward when it is determined that the length of stay of the patient is greater than the first time window, greater than the expected length of stay, and greater than the lower evaluation window and that the current TS lower bound value is less than the lower threshold.

12. The non-transitory machine-readable storage medium of claim 11, further comprising instructions for producing no recommendation regarding patient discharge when it is determined that the length of stay of the patient is not greater than a first time window, not greater than and expected length of stay, or not greater than a lower evaluation window.

13. The non-transitory machine-readable storage medium of claim 11, further comprising instructions for producing no recommendation regarding patient discharge when it is determined that that the current TS lower bound value is not less than the lower threshold.

14. The non-transitory machine-readable storage medium of claim 11, wherein the first time window has a value in the range of 8 to 24 hours.

15. The non-transitory machine-readable storage medium of claim 11, wherein the values of the first window, the lower evaluation window, and the lower threshold are determined by using machine learning techniques with patient training data.

16. The non-transitory machine-readable storage medium of claim 11, wherein the transition scores is further based upon diagnostic results, procedures performed, drugs consumed, medical images, or patient demographic information.

17. The non-transitory machine-readable storage medium of claim 11, wherein the patient vital signs include heart rate, respiration rate, peripheral capillary oxygen saturation (SpO2), blood pressure, and temperature.

18. The non-transitory machine-readable storage medium of claim 11, wherein calculating a transition score of a patient based upon patient vital sign information only occurs when the vital signs were measure within a specified recent period of time.

19. The non-transitory machine-readable storage medium of claim 11, further comprising instructions for:
determining if a length of stay of the patient is greater than a second time window and greater than an upper evaluation window;
determining if a current TS upper bound value is greater than an upper threshold; and
producing an indication that that the patient is to be evaluated for a step-up transition from the general ward when it is determined that the length of stay of the patient is greater than the second time window and greater than the lower evaluation window and that the current TS lower bound value is greater than the upper threshold.

20. The non-transitory machine-readable storage medium of claim 19, wherein the values of the second window, the upper evaluation window, and the upper threshold are determined by using machine learning techniques with patient training data.
